SEQUENCE LISTING (6 sequences)

SEQ ID NO: 1 - DNA, 35 bp, Artificial
  synthetic DNA
gcgctctagataagagaggtgacgtttgtcaggac

SEQ ID NO: 2 - DNA, 49 bp, Artificial
  synthetic DNA
cataggatccttagtggtggtggtggtggtgctcgtcacagaaaccgac

SEQ ID NO: 3 - DNA, 2848 bp, homo sapiens
  CDS (105)..(1688)
ggggttagcgcctgcgctctggacggctttggggcagggcagatttatatctgcgggggatcagctgacgctccgcattgcagactgcggagtcagacggcgctatgtacgccctcttcctcctggccagcctcctgggcgcggctctagccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaggatcagcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaactaggaggaggaatattccatcttggcagaaaccacagcattggtttttttctacttgtgtgtctgggggaatgaacgcacagatctgtttgactttgttataaaaatagggctcccccacctcccccatttctgtgtcctttattgtagcattgctgtctgcaagggagcccctagcccctggcagacatagctgcttcagtgccccttttctctctgctagatggatgttgatgcactggaggtcttttagcctgcccttgcatggcgcctgctggaggaggagagagctctgctggcatgagccacagtttcttgactggaggccatcaaccctcttggttgaggccttgttctgagccctgacatgtgcttgggcactggtgggcctgggcttctgaggtggcctcctgccctgatcagggaccctccccgctttcctgggcctctcagttgaacaaagcagcaaaacaaaggcagttttatatgaaagattagaagcctggaataatcaggctttttaaatgatgtaattcccactgtaatagcatagggattttggaagcagctgctggtggcttgggacatcagtggggccaagggttctctgtccctggttcaactgtgatttggctttcccgtgtctttcctggtgatgccttgtttggggttctgtgggtttgggtgggaagagggccatctgcctgaatgtaacctgctagctctccgaaggccctgcgggcctggcttgtgtgagcgtgtggacagtggtggccgcgctgtgcctgctcgtgttgcctacatgtccctggctgttgaggcgctgcttcagcctgcacccctcccttgtctcatagatgctccttttgaccttttcaaataaatatggatggcgagctcctaggcctctggcttcctggtagagggcggcatgccgaagggtctgctgggtgtggattggatgctggggtgtgggggttggaagctgtctgtggcccacttgggcacccacgcttctgtccacttctggttgccaggagacagcaagcaaagccagcaggacatgaagttgctattaaatggacttcgtgatttttgttttgcactaaagtttctgtgatttaacaataaaattctgttagccagaaaaaaaaaaaaaaaaaa

SEQ ID NO: 4 - protein, 527 aa, homo sapiens
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 5 - DNA, 246 bp, homo sapiens
  CDS (1)..(246)
ggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaggatcagcaacccaaggagatctgtgcgctggttgggttctgtgatgag

SEQ ID NO: 6 - protein, 82 aa, homo sapiens
GDVCQDCIQMVTDIQTAVRTNSTFVQALVEHVKEECDRLGPGMADICKNYISQYSEIAIQMMMHMQDQQPKEICALVGFCDE

(19) United States
(12) Patent Application Publication
(10) Pub. No.: US 2014/0219988 A1
(43) Pub. Date: Aug. 7, 2014

(54) RRECOMBINANT HUMAN SAPOSIN B PROTEIN CONTAINING PHOSPHORYLATED GLUCOSE RING AND USE THEREOF

(71) Applicants: Hitoshi Sakuraba, Tokyo (JP); Tadayasu Togawa, Tokyo (JP); Takahiro Tsukimura, Tokyo (JP); Yasunori Chiba, Tsukuba-shi (JP); Toru Watanabe, Tsukuba-shi (JP); Ikuo Kawashima, Tokyo (JP)

(72) Inventors: Hitoshi Sakuraba, Tokyo (JP); Tadayasu Togawa, Tokyo (JP); Takahiro Tsukimura, Tokyo (JP); Yasunori Chiba, Tsukuba-shi (JP); Toru Watanabe, Tsukuba-shi (JP); Ikuo Kawashima, Tokyo (JP)

(73) Assignee: TOKYO METROPOLITAN INSTITUTE OF MEDICAL SCIENCE, Tokyo (JP)

(21) Appl. No.: 14/118,410

(22) PCT Filed: Nov. 21, 2011
(86) PCT No.: PCT/JP2011/077290

§ 371 Date: Mar. 7, 2014

(30) Foreign Application Priority Data
May 19, 2011 (JP) 2011-112875

(51) Int. Cl. A61K 38/17; C07K 14/47; A61K 38/47

(52) U.S. Cl. CPC A61K 38/1709; A61K 38/47; C07K 14/47
USPC 424/94.61; 530/395

(57) ABSTRACT

The present invention provides a means to ensure a further increase in the therapeutic effects provided by enzyme replacement therapy against lysosomal disease. The present invention is directed to a recombinant human saposin B protein containing phosphorylated carbohydrate chains, a lysosomal enzyme activator comprising such a recombinant protein, and a pharmaceutical composition for treatment of lysosomal disease, which comprises such a recombinant protein and a lysosomal enzyme, etc.

### TECHNICAL FIELD

The present invention relates to a recombinant human saposin B protein containing phosphorylated carbohydrate chains (e.g., mannose-6-phosphate) and use thereof. More specifically, the present invention relates to, e.g., a lysosomal enzyme activator or a pharmaceutical composition for treatment of lysosomal disease, each comprising such a recombinant protein.

### BACKGROUND ART

Lysosomes which are one of the intracellular organelles contain many acidic hydrolases, and these acidic hydrolases are responsible for degradation of proteins, lipids, sugars or composites thereof taken into lysosomes from the intracellular and extracellular environments. When genetic impairment occurs in these lysosomal enzymes or in factors involved in the activation and stabilization of these enzymes or factors involved in the mechanism for transport of these enzymes to lysosomes, enzymatic reactions do not proceed in lysosomes and their substrates are accumulated in cells to thereby cause a series of diseases, which are collectively called “lysosomal disease” (Leroy J. G. and DeMars R. I., Mutant Enzymatic and Cytological Phenotype in Cultured Human Fibroblasts, Science, 157, 804-806 (1967)). As to lysosomal disease, around 40 types are known in humans and are one of the groups of diseases important in the pediatric and internal medicine areas. To develop radical treatments for these diseases, attempts have been made with bone marrow transplantation and/or gene therapy, etc. Among them, enzyme replacement therapy is now used as the most effective treatment.

Enzyme replacement therapy is a method in which lysosomal disease patients are externally administered with their lost enzymes, whereby substrates accumulated in large amounts can be degraded to improve the clinical condition of the patients. Techniques reported to obtain enzymes for replacement therapy include purification from placentas, production in cultured cells (e.g., fibroblasts, melanoma cells), recombination in cultured cells (e.g., insect cells, Chinese hamster ovary (CHO) cells), collection from the milk of transgenic rabbits, and production in yeast cells. However, most of the currently marketed therapeutic agents for lysosomal disease are produced as recombinants by using CHO cells or human skin-derived fibroblasts as host cells.

Fabry disease (hereditary α-galactosidase A deficiency), a kind of lysosomal disease, is a hereditary disease on chromosome X, which causes a reduction in the activity of α-galactosidase (α-galactosidase A) and accumulation of its in vivo substrate globotriaosylceramide (Gb3, CTH (ceramide trihexoside)) in the body (Kenneth J. Dean et al., Fabry Disease, “Practical Enzymology of the Sphingolipidoses”, U.S.A., Aln R. Liss, Inc., 1997, p. 173-216). Patients with “classic” Fabry disease having a typical progression will develop pain in extremity, angiokeratoma and/or hypohidrosis during their preadolescent to adolescent period, and will further develop renal disorders and/or cardiovascular and cerebrovascular disorders with advancing age. In recent years, the presence of “atypical” Fabry disease has been known, which is relatively mild and involves myocardial disorder as a cardinal symptom from the mature period. Patients with such atypical Fabry disease receive attention because they are reported to be hidden in a group of patients who are falsely diagnosed as having “cardiomyopathy.”

On the other hand, patients with lysosomal disease including Fabry disease, metachromatic leukodystrophy and GM2 gangliosidosis are deficient in enzymes which degrade sphingolipids (mainly glycosphingolipids). Non-enzymatic glycoproteins are required for these enzymes to act on glycolipids, and these glycoproteins are referred to as sphingolipid activator proteins. Sphingolipid activator proteins include prosaposin, which is a precursor of a series of activator protein saposins, and GM2 activator protein. In the former, prosaposin receives limited hydrolysis to produce activator proteins, i.e., saposins A, B, C and D. Prosaposin is located on the 10th chromosome in humans and is translated as a 58 kDa prosaposin. The respective saposins share high homology in their amino acid sequences, but differ in their substrate specificity, and are found to recognize specific glycolipids and promote degradation thereof (Kolter, T. and Sandhoff, K., Lysosomal degradation of membrane lipids. FEBS Lett, 2010. 584(9): p. 1700-1712). Saposin A is required for the degradation of galactosylceramide by galactosylceramide-β-galactosidase, while saposin B causes the degradation of sulfatide by arylsulfatase A, the degradation of globotriaosylceramide and digalactosylceramide by α-galactosidase, as well as the degradation of ganglioside GM1 together with GM2 activator protein (Wilkening, G, et al., Degradation of membrane-bound ganglioside GM1. Stimulation by bis(monoacylglycero)phosphate and the activator proteins SAP-B and GM2-AP. J Biol Chem, 2000. 275(46): p. 35814-35819). Saposin C causes the degradation of glucosylceramide by glucosylceramide-β-glucosidase, while saposin D causes the in vitro degradation of ceramide by acidic ceramidase. Although prosaposin deficiency is very rare, if there is a mutation in a gene encoding prosaposin, the above enzymatic reactions can no longer occur, thereby resulting in serious symptoms.

The crystal structure of saposin B has been reported (Ahn, V. E., et al., Crystal structure of saposin B reveals a dimeric shell for lipid binding. Proc Natl Acad Sci USA, 2003. 100(1): p. 38-43), and recent studies have also reported that saposin B facilitates recognition between CD1d and NKT cells (Yuan, W., et al., Saposin B is the dominant saposin that facilitates lipid binding to human CD1d molecules. Proc Natl Acad Sci USA, 2007. 104(13): p. 5551-5556). As described above, saposin B is produced from prosaposin, and hence the deficiency of saposin B alone is very rare. However, there are also patients who are deficient in saposin B alone due to mutation-induced exon skipping, and these patients are known to have a clinical condition like metachromatic leukodystrophy, a kind of lysosomal disease (Sun, Y., et al., Neurological deficits and glycosphingolipid accumulation in saposin B deficient mice. Hum Mol Genet, 2008. 17(15): p. 2345-2356).

Although enzyme replacement therapy has already been used in Fabry disease and other cases, this therapy has problems in 1) requiring high dose administration, 2) involving a concern about antigenicity, and 3) requiring high treatment costs for the reasons such as low uptake efficiency into lysosomes due to low content of lysosomal enzymes having phosphate-added carbohydrate chains, as well as low productivity and high culture costs. To solve these problems, there has been reported a method in which genetically modified yeast is used to increase the content of mannose-6-phosphate in the carbohydrate chains of α-galactosidase (JP 2002-369692 A). Moreover, in terms of reducing side effects, there has been developed a new enzyme for replacement therapy whose substrate specificity is altered by introducing a mutation into the active center of an enzyme similar in both primary structure and higher order structure (WO2008/143354).

### SUMMARY OF THE INVENTION

Under these circumstances, there has been a demand for the development of a means or method to ensure a further increase in the therapeutic effects provided by enzyme replacement therapy against lysosomal disease.

The present invention has been made in consideration of the above situation and aims to provide a recombinant human saposin B protein containing phosphorylated carbohydrate chains, a lysosomal enzyme activator, a pharmaceutical composition for treatment of lysosomal disease and so on, as shown below.

(1) A recombinant human saposin B protein containing phosphorylated carbohydrate chains.

In this recombinant protein, examples of phosphorylated carbohydrate chains include mannose-6-phosphate.

(2) A lysosomal enzyme activator, which comprises the recombinant protein according to (1) above.

In this activator, examples of a lysosomal enzyme include those for enzyme replacement therapy, more specifically at least one selected from the group consisting of α-galactosidase, arylsulfatase A, sialidase, acidic sphingomyelinase and β-galactosidase, with α-galactosidase being particularly preferred.

(3) A pharmaceutical composition for treatment of lysosomal disease, which comprises a lysosomal enzyme and/or a gene encoding the enzyme and the recombinant protein according to (1) above and/or a gene encoding the protein.

In particular, the composition according to (3) above may preferably be exemplified by a pharmaceutical composition for treatment of lysosomal disease, which comprises a lysosomal enzyme and the recombinant protein according to (1) above.

In the composition according to (3) above, examples of a lysosomal enzyme include those for enzyme replacement therapy, more specifically at least one selected from the group consisting of α-galactosidase, arylsulfatase A and β-galactosidase. Likewise, the lysosomal disease may be exemplified by at least one selected from the group consisting of Fabry disease, sialidosis, metachromatic leukodystrophy, saposin B deficiency and GM1 gangliosidosis. Above all, it is preferred that the lysosomal enzyme is α-galactosidase and the lysosomal disease is Fabry disease.

(4) A pharmaceutical composition for treatment of lysosomal disease, which comprises the recombinant protein according to (1) above and/or comprises a gene encoding the recombinant protein according to (1) above.

In the composition according to (4) above, the lysosomal disease may be exemplified by saposin B deficiency.

### BRIEF DESCRIPTION OF THE DRAWINGS

FIG. 1 shows the results of Western blot analysis obtained with the culture supernatant of the strain TRY131.

FIG. 2 shows the results of Western blot analysis obtained with the culture supernatant of the strain TRY144.

FIG. 3 shows the results of lectin blot analysis performed on saposin B purified from the strain TRY144. More specifically, mannosidase-untreated saposin B was electrophoresed in the left lane and mannosidase-treated saposin B was electrophoresed in the right lane, followed by lectin blot analysis.

FIG. 4 shows the effect of saposin B to induce the degrading activity of α-galactosidase on globotriaosylceramide (Gb3) in vitro. More specifically, this figure shows the analysis results of Gb3 by thin-layer chromatography (detection results of Gb3 and degraded products thereof at 37° C. after 24 hours). The lanes in the figure are as follows, respectively.

Lane Stds Standards of ceramide monohexoside (CMH), ceramide dihexoside (CDH), Gb3 and globotetraosylceramide (Gb4)

Lane 1: Gb3 standard

Lane 2: Gb3 alone (in the absence of enzyme (α-galactosidase))

Lane 3: Gb3+taurodeoxycholate (TDC)

Lane 4: Gb3+saposin B

Lane 5: Gb3+enzyme

Lane 6: Gb3+enzyme+TDC

Lane 7: Gb3+enzyme+saposin B

FIG. 5 shows the degrading effect on globotriaosylceramide (Gb3) accumulated in the kidney upon simultaneous administration of saposin B and enzyme (α-galactosidase) in mice. More specifically, this figure shows the analysis results of kidney Gb3 by thin-layer chromatography. The lanes in the figure are as follows, respectively.

Lane Stds: Standards of CMH, CDH, Gb3 and Gb4

Lane 1: Gb3 standard

Lane 2: Untreated wild-type mouse kidney

Lane 3: Untreated Fabry disease model mouse kidney

Lane 4: Fabry disease model mouse kidney after administration of enzyme alone

Lane 5: Fabry disease model mouse kidney after simultaneous administration of enzyme and saposin B

Lane 6: Fabry disease model mouse kidney after administration of saposin B alone

### DESCRIPTION OF EMBODIMENTS

The recombinant vaccinia virus of the present invention and use thereof will be described in more detail below. The scope of the present invention is not limited by the following description, and any embodiments other than those illustrated below may also be carried out with appropriate modifications without departing from the spirit of the invention. It should be noted that this specification incorporates the specification of Japanese Patent Application No. 2011-112875 (filed on May 19, 2011) in its entirety, based on which the present application claims priority. Moreover, all publications cited herein, including patent documents, non-patent documents and others, are incorporated herein by reference.

### 1. Summary of the Present Invention

As a strategy to solve the above conventional problems in enzyme replacement therapy, the inventors of the present invention have focused on the administration of saposin B, which is a sphingolipid activator protein, in combination with a lysosomal enzyme for replacement therapy. In this case, it is necessary to express saposin B on a large scale. Large-scale expression systems for saposin B have already been reported in E. coli or methanol yeast, but these systems are each intended for expression of proteins for use in structural analysis, but not intended for protein expression for combined administration with enzymes in enzyme replacement therapy. For this reason, the expressed proteins are not tested for their actual uptake into cells, and the efficiency of their uptake into cells is also low, so that the expressed proteins are not suitable for enzyme replacement therapy based on combined administration with enzymes.

As a result of extensive and intensive efforts, the inventors of the present invention have succeeded in obtaining saposin B which contains phosphorylated carbohydrate chains (more specifically mannose-6-phosphate), and have found that the combined administration of this saposin B and a lysosomal enzyme (e.g., α-galactosidase) allows more efficient degradation of globotriaosylceramide (Gb3) accumulated in cells. This finding led to the completion of the present invention.

### 2. Recombinant Human Saposin B Protein Containing Phosphorylated Carbohydrate Chains

The recombinant human saposin B protein of the present invention is a recombinant human saposin B protein containing phosphorylated carbohydrate chains, as described above.

Such phosphorylated carbohydrate chains are more specifically exemplified by mannose-6-phosphate. Moreover, although the recombinant human saposin B protein has no particular limitation on the content of phosphorylated carbohydrate chains among all carbohydrate chains in its molecule (i.e., the content as the number of carbohydrate chains), the content is preferably 5% or more, more preferably 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, or 85% or more, even more preferably 90% or more, and particularly preferably 91% or more, 92% or more, 93% or more, 94% or more, 95% or more, 96% or more, 97% or more, 98% or more, or 99% or more.

The recombinant human saposin B protein of the present invention also retains the properties as a sphingolipid activator protein and is excellent in the ability to activate lysosomal enzymes. Thus, such a recombinant protein is also useful as an activator of lysosomal enzymes. Namely, the present invention also provides a lysosomal enzyme activator comprising such a recombinant protein.

Lysosomal enzymes to be activated as above are not limited in any way, and preferred examples include α-galactosidase, arylsulfatase A, sialidase (e.g., acidic sialidase), acidic sphingomyelinase and β-galactosidase, with α-galactosidase being particularly preferred.

Moreover, the recombinant human saposin B protein of the present invention is a protein containing phosphorylated carbohydrate chains, as described above, and is excellent by itself in the uptake efficiency into cells. Thus, for example, when used alone or together with lysosomal enzymes for enzyme replacement therapy during this therapy, the recombinant human saposin B protein can also enhance the degrading activity of the lysosomal enzymes on their substrates in cells which constitute damaged tissues due to lysosomal disease. In these cases, lysosomal enzymes to be targeted by the above enzyme replacement therapy are not limited in any way, and preferred examples include α-galactosidase, arylsulfatase A and β-galactosidase, with α-galactosidase being particularly preferred. Likewise, lysosomal disease to be targeted by the above recombinant human saposin B is not limited in any way, and preferred examples include Fabry disease, sialidosis, metachromatic leukodystrophy, saposin B deficiency and GM1 gangliosidosis, with Fabry disease being particularly preferred. In particular, when the recombinant human saposin B is used alone, a preferred target disease is saposin B deficiency.

The recombinant human saposin B protein of the present invention is not limited in any way as long as it contains phosphorylated carbohydrate chains, as described above, and it is intended to also encompass, for example, a protein which consists of an amino acid sequence with deletion, substitution or addition of one or several amino acids in the amino acid sequence of the recombinant protein and which has the activity to activate lysosomal enzymes. It should be noted that wild-type human saposin B is produced by limited hydrolysis of wild-type human prosaposin and the amino acid sequence of this human saposin B (SEQ ID NO: 6) consists of amino acids 195 to 276 in the amino acid sequence of human prosaposin (SEQ ID NO: 4). Information of the above amino acid sequence of human prosaposin (SEQ ID NO: 4) and a nucleotide sequence encoding this sequence (SEQ ID NO: 3) is published, for example, under “Accession number: NP_{—}001035930” and “Accession number: NM_{—}001042465,” respectively, in the GenBank database (http://www.ncbi.nlm.nih.gov/genbank/) provided by the National Center for Biotechnology Information (NCBI). Likewise, the above amino acid sequence of human saposin B (SEQ ID NO: 6) is published under “Accession number: NP_{—}001035930” in the GenBank database, and a nucleotide sequence encoding this amino acid sequence (SEQ ID NO: 5) consists of nucleotides 687 to 932 in the above nucleotide sequence shown in SEQ ID NO: 3.

In the context of the present invention, the above “amino acid sequence with deletion, substitution or addition of one or several amino acids” is preferably, for example, an amino acid sequence with deletion, substitution or addition of about 1 to 10 amino acids, preferably about 1 to 5 amino acids, provided that it is preferable to minimize mutation (deletion, substitution or addition) from occurring in an amino acid residue serving as an N-type carbohydrate chain binding site in the amino acid sequence of wild-type human saposin B protein. Such an amino acid residue may be exemplified by amino acid residue 21 (asparagine (Asn: N)) in the above amino acid sequence shown in SEQ ID NO: 6 (i.e., an amino acid residue corresponding to the position of amino acid 215 in the amino acid sequence shown in SEQ ID NO: 4).

In the present invention, the activity to activate lysosomal enzymes may be detected or measured, for example, on the basis of the procedures described later in Example 4.

A gene encoding the recombinant human saposin B protein of the present invention is not limited to DNA comprising the above nucleotide sequence shown in SEQ ID NO: 5, and it also encompasses DNAs which are hybridizable under stringent conditions with DNA consisting of a nucleotide sequence complementary to DNA comprising the above nucleotide sequence and which encode a protein having the activity to activate lysosomal enzymes.

Such DNAs may be obtained from cDNA and genomic libraries by known hybridization techniques (e.g., colony hybridization, plaque hybridization, Southern blotting) using DNA comprising the nucleotide sequence shown in SEQ ID NO: 5 or DNA consisting of a nucleotide sequence complementary thereto or a fragment thereof as a probe. Any library may be used for this purpose, including those prepared in a known manner or commercially available cDNA and genomic libraries.

As to detailed procedures for hybridization, reference may be made, as appropriate, to Molecular Cloning, A Laboratory Manual 2nd ed. (Cold Spring Harbor Laboratory Press (1989).

The term “stringent conditions” in hybridization is intended to mean conditions for use in the washing step after hybridization, i.e., conditions including a buffer's salt concentration of 15 to 330 mM and a temperature of 25° C. to 65° C., preferably a salt concentration of 15 to 150 mM and a temperature of 45° C. to 55° C. More specifically, such conditions may be 80 mM and 50° C., by way of example. Further, in addition to such conditions of salt concentration and temperature, various other conditions such as probe concentration, probe length, reaction time and so on may also be taken into consideration in determining appropriate conditions.

DNA which is hybridizable is preferably a nucleotide sequence sharing a homology of at least 40% or more, more preferably 60% or more, 70% or more, 80% or more, 90% or more, particularly preferably 95% or more, 96% or more, 97% or more, 98% or more, and most preferably 99% or more with the nucleotide sequence shown in SEQ ID NO: 5.

Further, the above DNA preferably has no mutation (deletion, substitution or addition) in nucleotides representing a codon of the above amino acid residue serving as an N-type carbohydrate chain binding site, in comparison with the nucleotide sequence shown in SEQ ID NO: 5.

Moreover, particularly preferred as the above DNA is, for example, DNA consisting of a nucleotide sequence which is not completely identical with the nucleotide sequence shown in SEQ ID NO: 5, but its translated amino acid sequence is completely identical with that of the nucleotide sequence shown in SEQ ID NO: 5 (i.e., DNA carrying a silent mutation(s)).

The gene encoding the recombinant human saposin B protein of the present invention has no particular limitation on codons corresponding to the respective translated amino acids, and hence may comprise DNA representing codons commonly used (preferably frequently used) in humans or other mammals upon transcription or may comprise DNA representing codons commonly used (preferably frequently used) in microorganisms (e.g., E. coli or yeast) or plants, etc., upon transcription.

To express the recombinant human saposin B protein of the present invention, the above gene of the present invention should first be integrated into an expression vector to construct a recombinant vector. In this case, the gene to be integrated into an expression vector may optionally be modified in advance to have a transcription promoter, an SD sequence (if the host is a prokaryotic cell) and a Kozak sequence (if the host is an eukaryotic cell) linked to the upstream of the gene and/or a terminator linked to the downstream of the gene. In addition, other elements such as an enhancer, a splicing signal, a poly(A) addition signal, a selection marker and so on may also be linked in advance. It should be noted that the above transcription promoter and other elements required for gene expression are not limited in any way, i.e., these elements may be contained initially in the gene or if these elements are contained originally in an expression vector, they may be used.

For integration of the gene into an expression vector, it is possible to use various known techniques based on gene recombination technology, such as those using restriction enzymes or those using topoisomerase. Moreover, the expression vector may be of any type (e.g., plasmid DNA, bacteriophage DNA, retrotransposon DNA, retrovirus vector, artificial chromosomal DNA) as long as it is capable of carrying a gene encoding the protein of the present invention. A vector suitable for host cells to be used may be selected and used as appropriate.

Subsequently, the recombinant vector constructed above is introduced into a host to obtain a transformant, which is then cultured to allow the protein of the present invention to be expressed. It should be noted that the term “transformant” in the context of the present invention refers to a host carrying a foreign gene introduced thereinto, and examples include a host engineered to carry a foreign gene by introduction of plasmid DNA or the like (transformation), and a host engineered to carry a foreign gene by infection with various viruses and phages (transduction).

Such a host is not limited in any way and may be selected as appropriate, as long as it is capable of expressing the protein of the present invention after introduction of the above recombinant vector. Examples include known hosts such as various animal cells (e.g., human and mouse cells), various plant cells, bacteria, yeast, plant cells and so on.

In cases where animal cells are used as a host, human fibroblasts, CHO cells, monkey COS-7 or Vero cells, mouse L cells, rat GH3, human FL cells or the like may be used, by way of example, and more preferred are CHO cells or human fibroblasts. Moreover, it is also possible to use insect cells such as Sf9 cells or Sf21 cells. In cases where bacteria are used as a host, E. coli, Bacillus subtilis or the like may be used, by way of example.

In cases where yeast is used as a host, Saccharomyces cerevisiae, Schizosaccharomyces pombe or the like may be used, by way of example.

In cases where plant cells are used as a host, tobacco BY-2 cells or the like may be used, by way of example.

Techniques for obtaining a transformant are not limited in any way and may be selected as appropriate in consideration of the combination between host and expression vector types. Preferred examples include electroporation, lipofection, heat shock method, PEG method, calcium phosphate method, DEAE dextran method, as well as infection with various viruses (e.g., DNA and RNA viruses).

In the resulting transformant, the codon types of the gene contained in the recombinant vector are not limited in any way and may be either identical with or different from the codon types in the host actually used.

In general, production of a desired protein may be accomplished by a method which involves the step of culturing a transformant as described above and the step of collecting the desired protein from the resulting cultured product.

More specifically, the recombinant human saposin B protein of the present invention may be produced, for example, by being expressed in a transformant of any yeast strain, preferably a methylotrophic yeast strain. Preferred examples of a methanol-assimilable yeast strain include Ogataea minuta, Pichia pastoris, Hansenula polymorpha, Candida boidinii and so on. As to detailed procedures for production, for example, an expression vector having an alcohol oxidase (AOX) promoter or an expression vector having a glycolytic promoter which is constitutively expressed is first selected and a gene encoding human saposin B protein is introduced downstream of such a promoter to thereby construct a recombinant expression vector. The constructed vector is introduced into the above yeast cells by various gene transfer methods such as electroporation and lithium acetate method to obtain transformed yeast cells. Then, an expression vector carrying a phosphorylated carbohydrate chain transferase control gene, preferably a mannose-6-phosphate transferase control gene (more specifically a homolog gene thereof) is introduced into the transformed yeast cells, e.g., by gene transfer methods as described above, such that the transformed yeast cells will express saposin B with phosphorylated carbohydrate chains, to thereby obtain re-transformed yeast cells. Subsequently, for example in the case of using a vector which has an AOX promoter as a promoter for saposin B expression, the resulting transformed yeast cells are induced and cultured in a methanol-containing medium, whereby human saposin B protein with phosphorylated carbohydrate chains can be collected from the resulting cultured product.

The term “cultured product” used here is intended to mean any of a culture supernatant, cultured cells, cultured microorganisms, or a homogenate of such cells or microorganisms. The above transformant may be cultured according to standard procedures used for host culture. A desired protein is accumulated in the above cultured product.

As a medium used for the above culture, any of various known natural and synthetic media may be used as long as it contains a carbon source, a nitrogen source, inorganic salts and others which can be assimilated by the host and it ensures efficient culture of the transformant.

During culture, to avoid loss of the recombinant vector contained in the transformant and loss of the gene encoding the desired protein, the transformant may be cultured under selection pressure. Namely, in cases where a drug resistance gene is used as a selection marker, the corresponding drug may be added to the medium. Alternatively, in cases where an auxotrophic complementary gene is used as a selection marker, the corresponding nutritional factor may be removed from the medium. For example, in the case of culturing human fibroblasts transduced with a vector containing G418 resistance gene, G418 (G418 sulfate) may be added during culture, if necessary.

In the case of culturing a transformant or the like which is transformed with an expression vector comprising an inducible promoter as a promoter, a preferred inducer (e.g., IPTG) may be added to the medium, if necessary.

The transformant may be cultured under any conditions which do not impair the productivity of the desired protein and the growth of the host, i.e., usually at 10° C. to 40° C., preferably at 20° C. to 37° C., for 5 to 100 hours. pH adjustment may be accomplished by using an inorganic or organic acid, an alkaline solution, etc. Culture techniques include solid culture, static culture, shaking culture, aerobic spinner culture, etc.

After culture, when the desired protein is produced within microorganisms or cells, these microorganisms or cells may be homogenized to thereby collect the desired protein. Techniques used for homogenization of microorganisms or cells include high pressure treatment with a French press or a homogenizer, ultrasonic treatment, grinding treatment with glass beads or the like, enzymatic treatment with lysozyme, cellulase, pectinase or the like, freezing-thawing treatment, treatment with a hypotonic solution, phage-induced bacteriolysis, etc. After homogenization, homogenized residues from the microorganisms or cells (including an insoluble fraction of the cell extract) may be removed, if necessary. Techniques for residue removal include centrifugation and filtration, by way of example, during which a flocculant, a filter aid or the like may optionally be used to increase the efficiency of residue removal. The supernatant obtained after residue removal is a soluble fraction of the cell extract, which can be used as a crude protein solution.

Alternatively, when the desired protein is produced within microorganisms or cells, these microorganisms or cells per se may be collected by centrifugation, membrane separation or other techniques and may be used directly without being homogenized.

In contrast, when the desired protein is produced outside microorganisms or cells, the cultured solution may be used directly or treated by centrifugation, filtration or other techniques to remove the microorganisms or cells. Then, the desired protein may optionally be collected from the cultured product, e.g., by extraction through ammonium sulfate precipitation, and may further be isolated and purified, if necessary, by dialysis or various chromatographic techniques (e.g., gel filtration, ion exchange chromatography, affinity chromatography).

The production yield of the protein obtained by culturing the transformant or the like can be confirmed by SDS-PAGE (polyacrylamide gel electrophoresis) or other techniques, for example, in units per cultured solution, per wet or dry weight of the microorganisms, per protein in the crude enzyme solution, etc.

In addition to such a transformant-based protein synthesis system as described above, production of the desired protein may also be accomplished by using a cell-free protein synthesis system in which no living cell is used.

A cell-free protein synthesis system refers to a system for synthesizing a desired protein in an artificial container (e.g., a test tube) using a cell extract. Cell-free protein synthesis systems available for use also include cell-free transcription systems in which DNA is used as a template to synthesize RNA.

In this case, the cell extract to be used is preferably derived from host cells as described above. As a cell extract, it is possible to use, for example, an extract from eukaryotic or prokaryotic cells, more specifically an extract of CHO cells, rabbit reticulocytes, mouse L-cells, HeLa cells, wheat germ, budding yeast, E. coli or the like. It should be noted that these cell extracts may be used in any state, i.e., may be concentrated or diluted before use or may be used directly.

Such a cell extract can be obtained, for example, by ultrafiltration, dialysis, polyethylene glycol (PEG) precipitation, etc.

Such cell-free protein synthesis may also be accomplished by using a commercially available kit. Examples include reagent kits PROTEIOS™ (Toyobo Co., Ltd., Japan) and TNT™ System (Promega), as well as synthesizers PG-Mate™ (Toyobo Co., Ltd., Japan) and RTS (Roche Diagnostics), etc.

The desired protein produced by cell-free protein synthesis may be purified by chromatography or other means selected as appropriate, as described above.

### 3. Pharmaceutical Composition for Treatment of Lysosomal Disease

As described above, the recombinant human saposin B protein containing phosphorylated carbohydrate chains of the present invention is excellent by itself in the uptake efficiency into cells constituting a target organ (damaged organ) and is capable of effectively activating lysosomal enzymes such as α-galactosidase. Thus, the recombinant human saposin B protein of the present invention can be used as an active ingredient in therapeutic agents for lysosomal disease such as Fabry disease. Namely, the present invention provides a pharmaceutical composition for treatment of lysosomal disease, which comprises a lysosomal enzyme and the above recombinant human saposin B protein containing phosphorylated carbohydrate chains. More specifically, such a pharmaceutical composition is preferably a pharmaceutical composition for enzyme replacement therapy against lysosomal disease. It should be noted that the same explanation as provided above can also apply to specific examples of lysosomal disease and lysosomal enzymes. On the other hand, in another embodiment of the present invention, the recombinant human saposin B protein containing phosphorylated carbohydrate chains of the present invention can also be used alone as an active ingredient in therapeutic agents for lysosomal disease, particularly therapeutic agents for saposin B deficiency. Namely, the present invention also provides a pharmaceutical composition for treatment of lysosomal disease (particularly saposin B deficiency), which comprises the above recombinant human saposin B protein containing phosphorylated carbohydrate chains.

The lysosomal enzyme and the recombinant human saposin B protein, each serving as an active ingredient in such a pharmaceutical composition, may be used in any state, i.e., may optionally be converted into various salt or hydrate forms or may be chemically modified as appropriate in consideration of storage stability (particularly activity maintenance) as a therapeutic agent.

The pharmaceutical composition may be of any form as long as the lysosomal enzyme and the recombinant human saposin B protein can be administered in combination upon actual use. Thus, the lysosomal enzyme and the recombinant human saposin B protein may be stored separately from each other until actual use. In the present invention, embodiments of combined administration between the lysosomal enzyme and the recombinant human saposin B protein include all possible embodiments, i.e., those where both are administered in a pre-mixed state (simultaneous administration), those where both are administered alternately, and those where one is administered and the other is then administered.

The pharmaceutical composition may further comprise additional ingredients in addition to the above active ingredients including the lysosomal enzyme. Examples of such additional ingredients include various pharmaceutical ingredients (e.g., pharmaceutically acceptable various carriers) which are required depending on the dosage regimen (usage form) of the pharmaceutical composition. These additional ingredients may be contained as appropriate within a range that does not impair the effects provided by the above active ingredients.

In the pharmaceutical composition, the mixing ratio of the active ingredients including the lysosomal enzyme, and the type and mixing ratio of additional ingredients may be determined as appropriate in accordance with or in consideration of procedures for preparation of known enzyme drugs for replacement therapy (particularly enzyme drugs for replacement therapy for treatment of Fabry disease).

The pharmaceutical composition may be administered in any dosage regimen, but it is usual to select parenteral dosage regimens including intravenous drip infusion. Formulations available for use in various dosage regimens including parenteral dosage regimens can be prepared in a standard manner by appropriate selection from among pharmaceutically commonly used excipients, fillers, extenders, binders, wetting agents, disintegrants, lubricants, surfactants, dispersants, buffering agents, preservatives, solubilizers, antiseptics, correctives, soothing agents, stabilizing agents, isotonizing agents and so on.

As to the dosage form of the pharmaceutical composition, intravenous injections (including drip infusions) are usually selected for use. For example, the pharmaceutical composition may be provided in the form of unit-dose ampules or multi-dose containers, etc.

The dose of the pharmaceutical composition may usually be set to a wide range as appropriate in consideration of, e.g., the mixing ratio and/or type of active ingredients in the formulation and by taking into account the age and body weight of a target (patient) to be administered, the type of disease, symptoms, as well as the route of administration, the frequency of administration, the period of administration and so on. For example, the frequency of administration may be set to once every 2 to 4 weeks, and the dose (per administration) may be set to, for example, an amount at which the above lysosomal enzyme serving as an active ingredient can be administered at about 0.1 to 10 mg/kg patient body weight, about 0.1 to 5 mg/kg patient body weight, or about 0.2 to 1 mg/kg patient body weight.

In the present invention, not only the lysosomal enzyme, but also the recombinant human saposin B protein of the present invention is used as an active ingredient in the pharmaceutical composition. Thus, the degrading activity of the enzyme on its substrate is enhanced in cells which constitute damaged organs in lysosomal disease patients, and hence even when the lysosomal enzyme per se is used in a smaller amount when compared to conventional cases, it is possible to obtain an enzyme replacement effect (therapeutic effect) that is equal to or greater than in conventional cases and is also possible to greatly reduce the physical, mental and economic burdens on the patients.

In addition to the above pharmaceutical composition which comprises the recombinant human saposin B protein containing phosphorylated carbohydrate chains of the present invention, the present invention may also provide a pharmaceutical composition for treatment of lysosomal disease such as Fabry disease which comprises a gene encoding the recombinant human saposin B protein, i.e., a gene therapy agent for lysosomal disease. Specific embodiments preferably include, for example, a pharmaceutical composition for treatment of lysosomal disease which comprises a gene encoding a lysosomal enzyme and the above gene encoding the recombinant human saposin B protein, as well as a pharmaceutical composition for treatment of lysosomal disease (particularly saposin deficiency) which comprises the above gene encoding the recombinant human saposin B protein (used alone). It should be noted that such a gene encoding a lysosomal enzyme can be prepared by known gene recombination technology or DNA synthesis techniques, etc., on the basis of the gene sequences of various lysosomal enzymes published in known databases (e.g., GenBank). Likewise, the above gene encoding the recombinant human saposin B protein can be prepared by known gene recombination technology or DNA synthesis techniques, etc., on the basis of the above nucleotide sequence shown in SEQ ID NO: 5.

When the above pharmaceutical composition is used as a gene therapy agent, techniques used for this purpose include direct administration by injection, as well as administration of a vector carrying the intended nucleic acid. Examples of the above vector include an adenovirus vector, an adeno-associated virus vector, a herpes virus vector, a vaccinia virus vector, a retrovirus vector and a lentivirus vector, etc. The use of these virus vectors allows efficient administration. It should be noted that a commercially available gene transfer kit (e.g., product name: AdenoExpress, Clontech) may also be used for this purpose.

Alternatively, when the pharmaceutical composition is used in a gene therapy agent, the composition may be introduced into phospholipid vesicles (e.g., liposomes) and the resulting vesicles may be administered. In this case, vesicles holding a desired gene are introduced into given cells by lipofection. The resulting cells are then administered, for example, into veins or arteries, etc. Such vesicles may also be administered topically to the tissue of a damaged organ due to lysosomal disease (e.g., Fabry disease). For example, when the pharmaceutical composition is administered to adults, the daily dose per patient body weight may be about 0.1 μg/kg to 1000 mg/kg or about 1 μg/kg to 100 mg/kg.

It should be noted that the present invention may also provide a pharmaceutical composition, in which the above two pharmaceutical compositions, i.e., the pharmaceutical composition comprising the recombinant human saposin B protein and the pharmaceutical composition serving as a gene therapy agent are used in combination. Further, the present invention may also provide a pharmaceutical composition for treatment of Fabry disease, in which the respective active ingredients in the above two pharmaceutical compositions are used in combination as appropriate. Pharmaceutical compositions intended for such combined use may be exemplified by pharmaceutical compositions (i) to (vii) shown below:

(i) a pharmaceutical composition for treatment of Fabry disease, which comprises a lysosomal enzyme and a gene encoding the recombinant human saposin B protein of the present invention;

(ii) a pharmaceutical composition for treatment of Fabry disease, which comprises a gene encoding a lysosomal enzyme and the recombinant human saposin B protein of the present invention;

(iii) a pharmaceutical composition for treatment of Fabry disease, which comprises a lysosomal enzyme, as well as the recombinant human saposin B protein of the present invention and a gene encoding the protein;

(iv) a pharmaceutical composition for treatment of Fabry disease, which comprises a gene encoding a lysosomal enzyme, as well as the recombinant human saposin B protein of the present invention and a gene encoding the protein;

(v) a pharmaceutical composition for treatment of Fabry disease, which comprises a lysosomal enzyme and a gene encoding the enzyme, as well as the recombinant human saposin B protein of the present invention;

(vi) a pharmaceutical composition for treatment of Fabry disease, which comprises a lysosomal enzyme and a gene encoding the enzyme, as well as a gene encoding the recombinant human saposin B protein of the present invention; and

(vii) a pharmaceutical composition for treatment of Fabry disease, which comprises a lysosomal enzyme and a gene encoding the enzyme, as well as the recombinant human saposin B protein of the present invention and a gene encoding the protein.

### 4. Method for Treatment of Lysosomal Disease

The present invention encompasses a method for treatment of lysosomal disease, characterized in that the above pharmaceutical composition is administered to a patient with lysosomal disease (e.g., a patient with Fabry disease), more specifically a lysosomal enzyme and the recombinant human saposin B protein of the present invention, which are active ingredients in the above pharmaceutical composition, are administered in combination to such a patient, or alternatively, the recombinant human saposin B protein of the present invention is administered alone to such a patient. Moreover, in cases where the above pharmaceutical composition is the gene therapy agent described above, the present invention more specifically encompasses a method for treatment of lysosomal disease, characterized in that a gene encoding a lysosomal enzyme and a gene encoding the recombinant human saposin B protein of the present invention are administered in combination to such a patient, or alternatively, a gene encoding the recombinant human saposin B protein of the present invention is administered alone to such a patient.

Further, the present invention may also provide a method for treatment of lysosomal disease, in which the above two pharmaceutical compositions, i.e., the pharmaceutical composition serving as a gene therapy agent and the pharmaceutical composition comprising the recombinant human saposin B protein are used in combination, as well as a method for treatment of lysosomal disease, in which the respective active ingredients in the above two pharmaceutical compositions are used in combination as appropriate. As to such combined use, reference may be made, as appropriate, to the embodiments (i) to (vii) shown above in section 3, etc.

Moreover, the present invention also encompasses the use of the above pharmaceutical composition for treatment of lysosomal disease, as well as the use of the above pharmaceutical composition for manufacture of a therapeutic agent for lysosomal disease. In these uses, the pharmaceutical composition to be used is intended to include all the embodiments of pharmaceutical compositions shown above in section 3.

In these methods for treatment and these uses, the administration mode and dose preferred for the pharmaceutical composition of the present invention are as shown above in section 3.

### EXAMPLES

The present invention will be further described in more detail by way of the following examples, which are provided for illustrative purposes only and are not intended to limit the scope of the present invention.

### Example 1

### Preparation of Strain Producing Human Saposin B (Ogataea minutaΔoch1 Δura3 ade1::ADE-AOXp-SAPB His6)

The gene sequence of human saposin B (SEQ ID NO: 5) is composed of nucleotides 687 to 932 in the gene sequence of its precursor human prosaposin (SEQ ID NO: 3; GenBank Accession number: NM_{—}001042465). Based on this nucleotide sequence information, the gene sequence of human saposin B was modified to have codons suitable for expression in Ogataea minuta (methylotrophic yeast) and then synthesized in its entirety.

The entire synthesized human saposin B gene sequence was used as a template in PCR with the following primers A and B which bind to 3′ and 5′ regions of the gene sequence, respectively.

Primer A:
(SEQ ID NO: 1)
5′-GCGCTCTAGATAAGAGAGGTGACGTTTGTCAGGAC-3′
Primer B:
(SEQ ID NO: 2)
5′-CATAGGATCCTTAGTGGTGGTGGTGGTGGTGCTCGTCACAGAAACCG
AC-3′

The amplified DNA fragment obtained by PCR was cleaved with restriction enzymes Xba I and BamH I, followed by integration into the Xba I and BamH I sites of plasmid pOMEA1-His6 carrying an ADE1 marker to thereby construct a human saposin B expression plasmid having Saccharomyces cerevisiae secretion signal sequence α-factor prepro at the N-terminal end and 6 residues of histidine at the C-terminal end.

This plasmid was linearized by being cleaved at the Not I site, and was used to transform Ogataea minuta strain TK5-3 by electroporation. After transformation, the strain was seeded on a plate of YPD medium (2% polypeptone, 1% yeast extract, 2% glucose) and cultured at 30° C. for 3 days to obtain a transformant. Genomic DNA was prepared from the transformant and analyzed by PCR to confirm that the human saposin B gene had been integrated into the chromosome. The resulting transformant was designated as strain TRY131.

Subsequently, the following operations were conducted to confirm human saposin B expression. After the strain TRY131 was cultured in 5 ml of YPD medium at 30° C. for 2 days, the cells were collected by centrifugation and the medium was replaced with BMMY medium (1% methanol/Day, 1.34% yeast nitrogen base, 1% yeast extract, 2% polypeptone, 0.1 mg/ml uracil, 0.1 M potassium phosphate buffer (pH 6.0)), followed by culture at 20° C. for 3 days. After completion of the culture, the culture supernatant was obtained by centrifugation. The culture supernatant was denatured with SDS sample buffer and then analyzed by Western blot analysis in a standard manner. For Western blot analysis, mouse anti-tetra-His antibody (QIAGEN) was used as a primary antibody, while anti-mouse IgG antibody alkaline phosphatase conjugate (Cell Signaling technology) was used as a secondary antibody. Detection was accomplished by using ECL Plus Western Blotting Detection Reagents (GE Healthcare) and a CDD camera in a LAS-1000 (GE Healthcare) instrument.

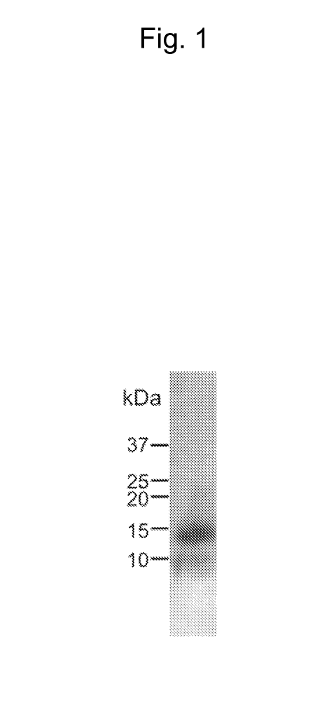

As a result, the control strain TK5-3 showed no signal, whereas the culture supernatant of the strain TRY131 was confirmed to show signals at the positions corresponding to molecular weights of approximately 9 kDa and 14 kDa (FIG. 1).

### Example 2

### Preparation of Strain Producing Human Saposin B with Phosphorylated Carbohydrate Chains (Ogataea minutaΔoch1 Δura3 Ade1::ADE-AOXp-SAPB His6 G418::AOXp-OmMNN4-1)

To prepare a strain producing human saposin B with phosphorylated carbohydrate chains, pOMEG1-OmMNN4 vector (see Akeboshi, H, Glycobiology., Vol. 19, p 1002-1009 (2009)) was used, which carries a gene for an Ogataea minuta homolog (OmMNN4-1) of Saccharomyces cerevisiae mannose-6-phosphate transferase control gene (ScMNN4). More specifically, this plasmid vector was linearized by being cleaved at the Not I site, and the resulting linearized vector was used to transform the strain TRY131 by electroporation. After transformation, the strain was seeded on a plate of YPD medium containing antibiotic G418 (200 μg/ml) and cultured at 30° C. for 3 days to obtain a transformant. Genomic DNA was extracted from the resulting transformant and analyzed by PCR to confirm that the OmMNN4-1 gene had been integrated under AOXp on the chromosome. This transformant was designated as strain TRY144.

Subsequently, after the above strain TRY144 was cultured in 5 ml of YPD medium at 30° C. for 2 days, the cells were collected by centrifugation and the medium was replaced with BMMY medium, followed by culture at 20° C. for 3 days. After completion of the culture, the culture supernatant was obtained by centrifugation. The culture supernatant was denatured with SDS sample buffer and then analyzed by Western blot analysis in the same manner as shown in Example 1.

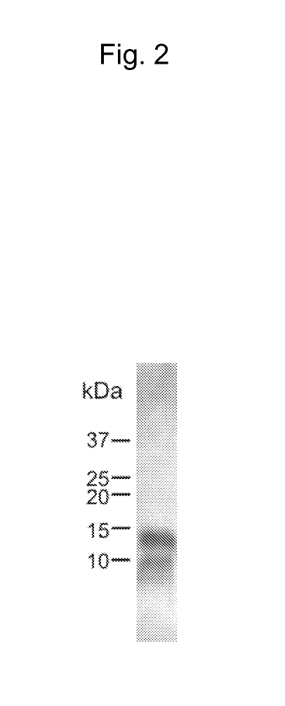

As a result, as in the case of the parent strain TRY131, the culture supernatant of the strain TRY144 was also confirmed to show signals at the positions corresponding to molecular weights of approximately 9 kDa and 14 kDa (FIG. 2).

### Example 3

### Purification of Human Saposin B from the Culture Supernatant of Strain Carrying Human Saposin B Gene, and Carbohydrate Chain Structural Analysis of Purified Enzyme

For large-scale production of human saposin B protein, the culture was scaled up. First, the strain TRY144 obtained in Example 2 was preliminarily pre-cultured in 10 ml of YPD medium at 30° C. for 2 days, and then pre-cultured at 30° C. for 2 days in 200 ml of YPD medium, followed by culture at 25° C. in 4 L of rich BMGY medium (1% yeast extract, 6% polypeptone, 100 mM potassium phosphate buffer (pH 6.0), 1.34% yeast nitrogen base, 4% glycerol). The end point of glycerol consumption was monitored using the value of dissolved oxygen concentration (DO) as an indicator, and methanol addition was initiated with increase in the DO value. Concurrently, an oxygen generator was used to blow an oxygen gas into the medium to thereby keep the DO value at 5.0 ppm. Moreover, the culture temperature was reduced to 20° C., starting from 15 hours before methanol addition, and methanol was added continuously (about 1.5%/day). After 72 hours, the culture supernatant was collected.

The culture supernatant was adjusted to pH 7.4 and then centrifuged to remove precipitates, and further passed through 0.4 μm and 0.22 μm filters to remove insoluble substances. This culture supernatant was subjected to HisPrep FF 16/10 (GE Healthcare) and washed with 50 mM sodium phosphate buffer (pH 7.4), 300 mM NaCl and 20 mM imidazole, followed by elution with 50 mM sodium phosphate buffer (pH 7.4), 300 mM NaCl and 500 mM imidazole. The eluted fraction was denatured with SDS sample buffer and then analyzed by Western blot analysis in the same manner as shown in Example 1 to collect a fraction showing strong signals, which was used as a crude protein solution.

After buffer replacement with 25 mM MES buffer (pH 6.0), the collected fraction was subjected to HiPrep Q FF 16/10 and washed, followed by gradient elution with 25 mM MES buffer (pH 6.0) and 1 M NaCl. The eluted fraction was denatured with SDS sample buffer and then analyzed by Western blot analysis in the same manner as shown in Example 1 to collect a fraction showing strong signals. This collected fraction was used as a purified standard for carbohydrate chain structural analysis.

The resulting human saposin B was enzymatically treated to cleave asparagine-linked carbohydrate chains. After buffer replacement with MilliQ water, the sample was lyophilized and dissolved in 10 μl of N-glycosidase F buffer (0.5 M Tris-HCl buffer (pH 8.6) containing 0.5% SDS and 0.75% 2-mercaptoethanol), followed by boiling for 5 minutes. After cooling to room temperature, 10 μl of 5.0% Nonidet P-40, 26 μl of H₂O and 4 μl of N-glycosidase F (Roche) were added for treatment at 37° C. for 16 hours. After being enzymatically treated, the sample was supplemented with 100% ethanol and allowed to stand at −20° C. for 20 minutes, followed by centrifugation to collect the supernatant, which was then dried for use as a carbohydrate chain preparation.

To fluorescently label the resulting carbohydrate chains (pyridylamination; hereinafter referred to as “PA modification”), the following operations were conducted. After the carbohydrate chain preparation was concentrated to dryness, 40 μl of coupling reagent (40 mg of 2-aminopyridine dissolved in 13.4 μl of acetic acid) was added, and the resulting mixture was sealed and treated at 90° C. for 60 minutes. After cooling to room temperature, 40 μl of reducing reagent (8 mg of borane-dimethylamine complex dissolved in 40 μl of acetic acid) was added, and the mixture was sealed and treated at 80° C. for 60 minutes. After the reaction, 120 μl of MilliQ water was added. The reaction mixture was extracted seven times with 200 μl of a mixed solvent composed of phenol:chloroform:isoamyl alcohol=25:24:1 (v/v/v), and then extracted once with 200 μl of a mixed solvent composed of chloroform:isoamyl alcohol=24:1 (v/v) to remove unreacted 2-aminopyridine. The carbohydrate chain fraction was filtered through a 0.22 μm filter for use as a PA-modified oligosaccharide preparation.

Analysis of phosphorylated carbohydrate chains was performed by using HPLC (see Akeboshi, H, Glycobiology., Vol. 19, p 1002-1009 (2009)). The sample was fractionated with a Shodex Asahipak NH2P-50 4E (Showa Denko KK, Japan) column, and each fraction was dried and then dissolved in MilliQ water, followed by analysis with TSK-gel Amide-80 (Tosoh Corporation, Japan).

The results obtained are shown in Table 1. In the strain TRY131, neutral carbohydrate chains composed of high mannose type carbohydrates constituted 73.1%, while acidic carbohydrate chains composed of phosphorylated carbohydrate chains constituted 26.8%. On the other hand, in the strain TRY144, the ratio of acidic carbohydrate chains was 95.9%, thus indicating that the ratio of phosphorylated carbohydrate chains (mannose-6-phosphate) among all carbohydrate chains was very high.

TABLE 1
Carbohydrate chain structure of saposin B

| | Strain TRY131 | Strain TRY144 |
|---|---|---|
| Neutral carbohydrate chains | 73.1 | 4.1 |
| Acidic carbohydrate chains | 26.8 | 95.9 |
| Monophosphate | 26.4 | 14.4 |
| Diphosphate | 0.4 | 81.5 |

To expose mannose-6-phosphate on the carbohydrate chains added to human saposin B, the crude protein solution was subjected to buffer replacement with 10 mM HEPES (pH 7.0) and 1 mM CaCl₂, followed by mannosidase treatment with SO-5-derived mannosidase (see Chiba, Y, Glycobiology., Vol. 12, p 821-828 (2002)).

After being adjusted to pH 7.4, the mannosidase-treated solution was subjected to HisPrep HP (GE Healthcare), washed with 50 mM sodium phosphate buffer (pH 7.4), 300 mM NaCl and 20 mM imidazole, and then eluted with 50 mM sodium phosphate buffer (pH 7.4), 300 mM NaCl and 500 mM imidazole. The eluted fraction was denatured with SDS sample buffer and then analyzed by Western blot analysis in the same manner as shown in Example 1 to collect a fraction showing strong signals.

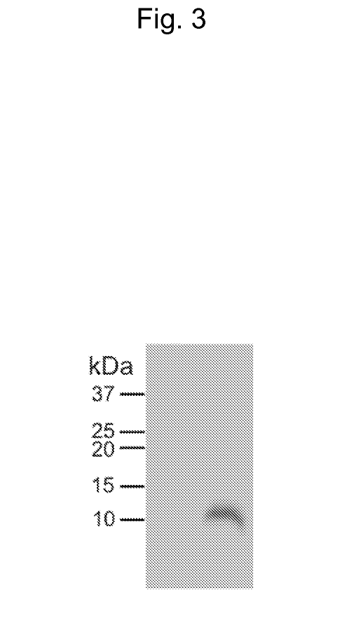

After buffer replacement with 20 mM Tris-HCl (pH 7.4), 500 mM NaCl, 1 mM MnCl₂ and 1 mM CaCl₂, the collected fraction was subjected to HiTrap Con A 4B (GE Healthcare), washed with 20 mM Tris-HCl (pH 7.4), 500 mM NaCl, 1 mM MnCl₂ and 1 mM CaCl₂, and then eluted with 20 mM Tris-HCl (pH 7.4), 500 mM NaCl and 500 mM glucose. The eluted fraction was denatured with SDS sample buffer and then analyzed by Western blot analysis in the same manner as shown in Example 1 to collect a fraction showing strong signals. The collected fraction was subjected to buffer replacement with PBS for use as a purified standard of human saposin B. Moreover, to confirm the exposure of mannose-6-phosphate on the carbohydrate chains, lectin blot analysis was performed using domain 9 of the cation-independent mannose-6-phosphate receptor (see Akeboshi, H, Appl Environ Microbiol., Vol. 73, p 4805-4812 (2007)). Saposin B was electrophoresed on an SDS-polyacrylamide gel and then transferred onto a PVDF membrane and blocked with skimmed milk, followed by lectin blot using a recombinant protein of domain 9 of the cation-independent mannose-6-phosphate receptor as a lectin. After washing with PBS, analysis was performed using antibodies for detection, i.e., mouse anti-tetra-His antibody (QIAGEN) as a primary antibody and anti-mouse IgG antibody alkaline phosphatase conjugate (Cell Signaling technology) as a secondary antibody. The results obtained are shown in FIG. 3. Although no signal was confirmed in mannosidase-untreated saposin B, signals were observed in mannosidase-treated saposin B, thus confirming that phosphorylated carbohydrate chains recognizable by the mannose-6-phosphate receptor were added to saposin B.

### Example 4

### Measurement of Saposin B-Induced In Vitro α-Galactosidase Activation

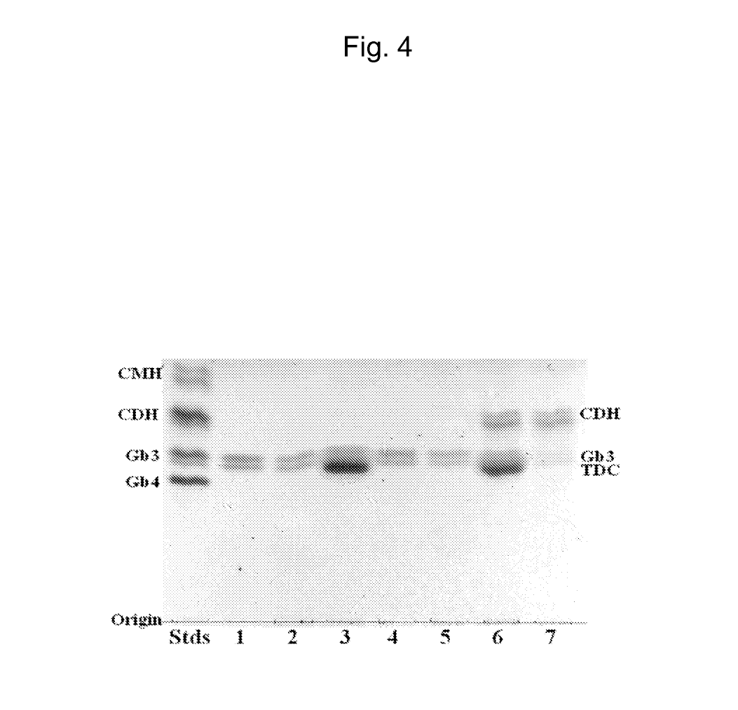

For use as a substrate, 10 μg of globotriaosylceramide (Gb3) was taken into a 1.5 ml Eppendorf tube and dried up. This tube was charged with 20 μl of 50 mM citrate-phosphate buffer (pH 4.6) and further with MilliQ water in a volume remaining after subtracting the volume of reagents to be added later, to give a final volume of 100 μl. The tube was fully shaken with a vortex mixer and then ultrasonicated for 20 seconds to form micelles. The tube was charged with 10 μl of 50 mM taurodeoxycholate serving as a solubilizer for use as a positive control or with 5 μl of saposin B (260 μg/ml) (saposin B produced in the above yeast strain (TRY144) (see Example 3)) for use as an analyte, followed by shaking. Subsequently, each tube was charged with 2 μl of an enzyme (α-galactosidase) solution (activity: 500 nm/h/ml) and then shaken. In an incubator at 37° C., the enzymatic reaction was continued until the predetermined time (24 hours in FIG. 4). 20 μl of methanol was added to stop the reaction, and the reaction solution was dried up under a nitrogen stream.

To the above tube, 50 μl of 0.3 M ammonium acetate buffer (pH 7.0) was added to dissolve the residue, and the resulting solution was loaded on to a Sep-Pak Vac 1 cc (50 mg) tC18 Cartridge (Waters), which had been equilibrated with PBS, for desalting purposes. The column was washed with MilliQ water and then eluted with methanol. This methanol-eluted fraction was dried up under a nitrogen stream. The final residue was dissolved by addition of 50 μl of a mixed solvent composed of chloroform:methanol:water=60:30:4.5 (v/v/v) for use as a sample for thin-layer chromatography (TLC) analysis.

Analysis of Gb3-containing glycolipids was performed by using TLC. On a TLC plate, the above sample was loaded in a volume of 10 μl (corresponding to 2 mg of Gb3). TLC was accomplished by using a mixed solvent composed of chloroform:methanol:0.22% aqueous CaCl₂=65:25:4 (v/v/v) as a developing solvent, and the developed plate was treated with orcinol-sulfuric acid reagent to develop color and then analyzed with a luminescent image analyzer LAS-4000 (Fuji Photo Film Co., Ltd., Japan) equipped with general-purpose analytical software Multi Gauge Ver3.X.

The results obtained are shown in FIG. 4. After 24 hours, the enzyme α-galactosidase) alone (Lane 5) showed no degradation of the substrate Gb3 (of course, taurodeoxycholate having no α-galactosidase activity (Lane 3) and saposin B alone (Lane 4) also showed no degradation of Gb3). The combination of taurodeoxycholate and the enzyme used as a positive control (Lane 6), and the combination of saposin B and the enzyme (Lane 7) showed a reduction in the substrate Gb3 and appearance of CDH (ceramide dihexoside), a degraded product of Gb3. This substrate degradation was increased in proportion to the amount of the enzyme or saposin B added to the reaction system and in proportion to the reaction time. This result indicated that saposin B used in this example induced α-galactosidase activation in vitro.

### Example 5

### Simultaneous Administration of Saposin B and α-Galactosidase Allows Effective Degradation of Gb3 Accumulated in the Kidneys of Fabry Disease Model Mice

Yeast-produced saposin B which had been confirmed to have α-galactosidase activity in vitro (i.e., saposin B purified in Example 3) was tested for its degrading effect on the substrate Gb3 accumulated in the kidney, which is a target organ of Fabry disease, by using Fabry disease model mice. The test groups prepared are as follows: the group of untreated wild-type mice (negative control), the group of Fabry disease mice (positive control), the group receiving 0.2 mg/kg α-galactosidase (product name: Replagal®, Shire HGT; the same applying hereinafter in this example), the group receiving 0.25 mg/kg saposin B, and the group receiving both 0.2 mg/kg α-galactosidase and 0.25 mg/kg saposin B. Mice in each group were administered five times via the tail vein at an interval of once a day. At 24 hours after the final administration, kidneys were excised from the mice for analysis of Gb3.

For analysis of kidney Gb3, first, the excised kidneys were each weighed and a homogenate was prepared from each kidney with 4 volumes of PBS and transferred to a screw-capped glass test tube, to which 2.7 volumes of methanol was then added dropwise under shaking, followed by shaking for several hours. Subsequently, 1.35 volumes of chloroform was added dropwise and the test tubes were shaken overnight. The test tubes were each centrifuged to collect the supernatant, and the centrifugal residue was mixed with 0.5 ml of MilliQ water and shaken. After 2.0 ml of a mixed solvent composed of chloroform:methanol=1:2 (v/v) was added dropwise, the test tubes were each shaken for several hours and then centrifuged to collect the supernatant. This procedure was repeated twice, and the collected supernatants were combined and dried up under a nitrogen stream. To the final residue, a mixed solvent composed of chloroform:methanol:water=60:30:4.5 (v/v/v) was added to give a TLC sample whose final concentration was 1 μl per 0.5 mg of organ wet weight. Analysis of Gb3 was performed in the same manner as shown in Example 4.

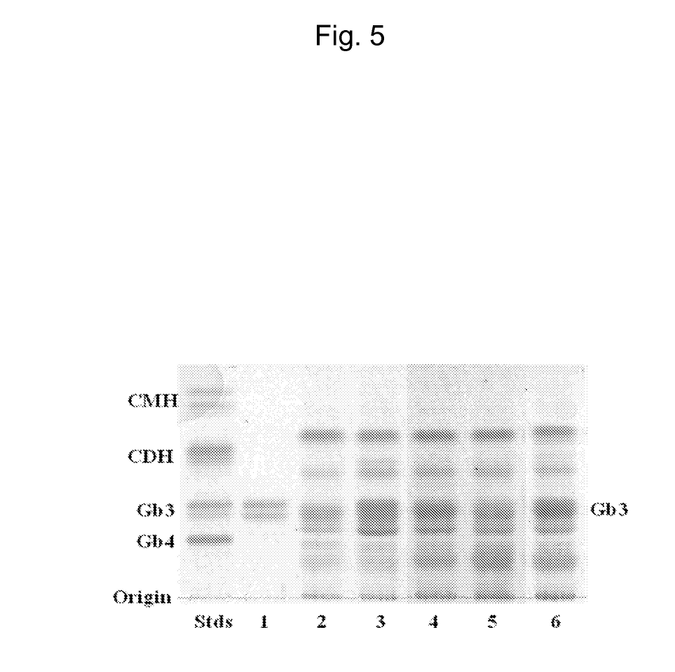

The results obtained are shown in FIG. 5. When compared to the kidneys of the untreated wild-type mouse group (Lane 2), the kidneys of the untreated Fabry disease mouse group (Lane 3) were found to have high Gb3 content. The kidneys of the group receiving α-galactosidase (Lane 4) showed a reduction in Gb3. However, the kidneys of the Fabry disease mouse group receiving saposin B alone (Lane 6) showed little reduction in Gb3. Further, the kidneys of the group receiving both α-galactosidase and saposin B (Lane 5) showed a significant reduction in Gb3, when compared to the kidneys of the group receiving the enzyme alone (Lane 4). These results indicated that simultaneous administration of α-galactosidase and saposin B enhanced the degrading effect on Gb3, when compared to administration of α-galactosidase alone. These results suggest that it is possible to expect a reduction in the total dose of lysosomal enzymes and prolongation of the administration interval in enzyme replacement therapy.

### INDUSTRIAL APPLICABILITY

The present invention enables the provision of a recombinant human saposin B protein containing phosphorylated carbohydrate chains modified from human saposin B, which is a sphingolipid activator protein, as a means to ensure further increase in the therapeutic effects provided by enzyme replacement therapy against lysosomal disease.

The recombinant human saposin B protein of the present invention allows effective activation of lysosomal enzymes for enzyme replacement therapy, and also provides a degrading effect on sphingolipids, which has not been achieved simply by conventional modifications to lysosomal enzymes, when administered to patients together with lysosomal enzymes during enzyme replacement therapy. Accordingly, the recombinant human saposin B protein of the present invention is very useful in that it enables the provision of a lysosomal enzyme activator having the above effects and a pharmaceutical composition for treatment of lysosomal disease, etc.

### SEQUENCE LISTING FREE TEXT

SEQ ID NO: 1: synthetic DNA

SEQ ID NO: 2: synthetic DNA

1. A recombinant human saposin B protein containing phosphorylated carbohydrate chains.

2. The recombinant protein according to claim 1, wherein the phosphorylated carbohydrate chains are composed of mannose-6-phosphate.

3. A lysosomal enzyme activator, which comprises the recombinant protein according to claim 1 or 2.

4. The activator according to claim 3, wherein the lysosomal enzyme is one for enzyme replacement therapy.

5. The activator according to claim 3, wherein the lysosomal enzyme is at least one selected from the group consisting of α-galactosidase, arylsulfatase A, sialidase, acidic sphingomyelinase and β-galactosidase.

6. The activator according to claim 3, wherein the lysosomal enzyme is α-galactosidase.

7. A pharmaceutical composition for treatment of lysosomal disease, which comprises a lysosomal enzyme and/or a gene encoding the enzyme and the recombinant protein according to claim 1 or 2 and/or a gene encoding the protein.

8. A pharmaceutical composition for treatment of lysosomal disease, which comprises a lysosomal enzyme and the recombinant protein according to claim 1 or 2.

9. The composition according to claim 7, wherein the lysosomal enzyme is one for enzyme replacement therapy.

10. The composition according to claim 7, wherein the lysosomal enzyme is at least one selected from the group consisting of α-galactosidase, arylsulfatase A and β-galactosidase.

11. The composition according to claim 7, wherein the lysosomal disease is at least one selected from the group consisting of Fabry disease, sialidosis, metachromatic leukodystrophy, saposin B deficiency and GM1 gangliosidosis.

12. The composition according to claim 7, wherein the lysosomal enzyme is α-galactosidase and the lysosomal disease is Fabry disease.

13. A pharmaceutical composition for treatment of lysosomal disease, which comprises the recombinant protein according to claim 1 or 2.

14. A pharmaceutical composition for treatment of lysosomal disease, which comprises a gene encoding the recombinant protein according to claim 1 or 2.

15. The composition according to claim 13, wherein the lysosomal disease is saposin B deficiency.
